(19) United States
(12) Patent Application Publication
(10) Pub. No.: US 2013/0280724 A1
(43) Pub. Date: Oct. 24, 2013

(54) METHODS FOR DIAGNOSING IMPENDING DIARRHEA

(71) Applicant: Nestec SA, (US)

(72) Inventors: Ziad S. Ramadan, Manchester, MO (US); Dorothy P. Laftamme, Floyd, VA (US); Hui Xu, Chesterfield, MO (US)

(73) Assignee: Nestec SA, Vevey (CH)

(21) Appl. No.: 13/851,421

(22) Filed: Mar. 27, 2013

Related U.S. Application Data
(60) Provisional application No. 61/622,765, filed on Apr. 11, 2012.

(51) Int. Cl. C12Q 1/68

(52) U.S. Cl. CPC C12Q 1/689
USPC 435/6.12

(57) ABSTRACT

The invention provides methods for diagnosing impending diarrhea in an asymptomatic animal. The methods comprise obtaining a fecal sample from the animal; identifying one or more microorganisms in the fecal sample; and diagnosing impending diarrhea in the animal based on the presence or absence of one or more of the microorganisms.

### CROSS REFERENCE TO RELATED APPLICATIONS

This application claims priority to U.S. Provisional Application No. 61/622,765 filed Apr. 11, 2012, the disclosure of which is incorporated herein by this reference.

### BACKGROUND OF THE INVENTION

1. Field of the Invention

The invention relates generally to methods for diagnosing diarrhea and particularly to methods for diagnosing impending diarrhea in an asymptomatic animal.

2. Description of the Related Art

Chronic diarrhea, loose watery stool that is present for three or more weeks, can be extremely unpleasant to both an animal and its caregiver. The condition is typically a nuisance but persistent diarrhea can lead to dehydration, weight loss, and nutrient deficiency due to lack of absorption, it can also lead to the development of poor hair coat and may affect appetite and activity levels thereby adversely affecting, the animal's health and wellness.

There are many different causes of chronic diarrhea. For example, some types of chronic diarrhea are caused by an infection such as would be caused by a parasite, a bacterial infection or a viral infection. Other cases of chronic diarrhea are caused by food sensitivities, medications, antibiotics, or other drugs ingested into the body. Still other cases of chronic diarrhea are caused by more serious medical conditions such as tumors, diabetes, thyroid and other endocrine diseases, food allergies, reduced blood flow into the intestine, colitis, and/or Crohn's disease. Yet, in many cases the cause of chronic diarrhea is unknown.

Currently, diarrhea is diagnosed after symptoms present. Current methods for diagnosing the cause of chronic diarrhea include fecal studies, such as flotation, smear and cytology, and zinc sulfate test, to search for intestinal parasites, protozoal parasites, and bacteria. Current methods for treating diarrhea often involve modifying the diet or administering drugs useful for treating parasites or commonly know pathogens associated with diarrhea. There are no known methods for diagnosing impending diarrhea in an asymptomatic animal. There is, therefore, a need for new methods for diagnosing impending diarrhea in asymptomatic animals.

### SUMMARY OF THE INVENTION

It is, therefore, an object of the invention to provide methods for diagnosing impending, diarrhea in an asymptomatic animal.

It is another object of the invention to provide methods for promoting the health and wellness of an animal.

These and other objects are achieved using methods for diagnosing impending diarrhea in an animal. The methods comprise obtaining a fecal sample from the animal; and identifying one or more microorganisms in the fecal sample; and diagnosing impending diarrhea in the animal based on the presence or absence of one or more of the microorganisms.

Other and further objects, features, and advantages of the invention will be readily apparent to those skilled in the art.

### DETAILED DESCRIPTION OF THE INVENTION

### Definitions

The term “animal” means any animal susceptible to diarrhea. An animal is “susceptible to” a disease or condition if the animal exhibits symptoms that indicate that the animal is likely to develop the condition or disease within the next 10 days.

The term “companion animal” means domesticated animals such as cats, dogs, rabbits, guinea pigs, ferrets, hamsters, mice, gerbils, horses, cows, goats, sheep, donkeys, pigs, and the like.

The term “health and/or wellness of an animal” means the complete physical, mental, and social well being of the animal, not merely the absence of disease or infirmity.

The terms “treating”, “treat”, and “treatment” embrace both preventative, i.e., prophylactic, and palliative treatment.

### The Invention

In one aspect, the invention provides methods for diagnosing impending diarrhea in an asymptomatic animal comprising obtaining a fecal sample from the animal; identifying one or more microorganisms in the fecal sample: and diagnosing impending diarrhea in the animal based on the presence or absence of one or more of the microorganisms. The animal is diagnosed to have impending diarrhea if (1) one or more microorganisms selected, from a first group consisting of Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri. Bulleidia p_{—}1630_c5, Parabacterides distasonis, Ruminococcus gnavus, Ruminococcus torques, Streptococcus minor, and Streptococcus suis are present in the feral sample, (2) one or more microorganisms selected from a second group consisting of Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri are absent from the fecal sample, or (3) combinations thereof. In preferred embodiments, the animal is diagnosed to have impending diarrhea if (1) two, three, four, five or more microorganisms from a first group consisting of Alistipes putredinis, Anaerobiospirillum succiniciproducens. Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri, Bulleidia p_{—}1630_c5, Parabacteroides distasonis, Ruminococcus gnavus, Ruminococcus torques, Streptococcus minor, and Streptococcus suis are present in the fecal sample, (2) two, three, four, five or more microorganism selected from a second group consisting of Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri are absent from the fecal sample, or (3) combinations thereof.

In certain preferred embodiments, the animal is diagnosed to have impending diarrhea if (1) at least three or more microorganisms selected from a first group consisting of Alistipes putredinis, Anaerobiospirillum succiniciproducens. Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri, Bulleidia p_{—}1630_c5, Parabacteroides distasonis, Ruminococcus gnavus, Ruminococcus torques. Streptococcus minor, and Streptococcus suis are present in the fecal sample or (2) at least three or more microorganisms selected from a second group consisting of Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri are absent from the fecal sample.

In certain embodiments, the microorganisms in the first group are selected from the group consisting of Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri, Bulleidia p_{—}1630_c5, Parabacteroides distasonis, Ruminococcus gnavus, Ruminococcus torques, Streptococcus minor, and Streptococcus suis. In other embodiments, the microorganisms in the first group are selected from the group consisting of Bacteroides coprocola, Clostridium perfringens, Clostridium spiroforme, Enterobacter hormaechei, Faecalibacterium prausnitzii, Helicobacter muridarum, Lactobacillus helveticus, p_{—}1630_c5, Ruminococcus guavus, Ruminococcus torques, Streptococcus minor, and Streptococcus suis, in other embodiments, the microorganisms in the first group are selected from the group consisting of Clostridium spiroforme and Streptococcus suis.

In certain embodiments, the microorganisms in the second group are selected from the group consisting of Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri. In other embodiments, the microorganisms in the second group are selected from the group consisting of Acidaminococcus fermentans, Campylobacter upsaliensis, Clostridium orbiseindens, Megasphaera elsdenii, and Prevotella copri. In other embodiments, the microorganisms M the second group are selected from the group consisting of Acidaminococcus fermentans and Campylobacter upsaliensis.

The inventions are based upon the discovery that certain microorganisms have been associated with the incidence of diarrhea and others microorganisms have been associated with the absence of diarrhea. These microorganisms are identified in Table I.

TABLE 1
Microorganisms Associated with Diarrhea

| Phylum | Class | Order | Family | Genus | Species |
|---|---|---|---|---|---|
| Actinobacteria | Actinobacteria | Coriobacteriales | Coriobacteriaceae | Collinsella | aerofaciens |
| | | | | Slackia | |
| Bacteroidetes | Bacteroidia | Bacteroidales | Bacteroidaceae | Bacteroides | coprocola |
| | | | | | coprophilus |
| | | | | | plebeius |
| | | | Porphyromonadaceae | Parabacteroides | distasonis |
| | | | Prevotellaceae | Prevotella | copri |
| | | | Rikenellaceae | Alistipes | putredinis |
| Firmicutes | Bacilli | Lactobacillales | Lactobacillaceae | Lactobacillus | helveticus |
| | | | | | saerimneri |
| | | | Streptococcaceae | Streptococcus | minor |
| | | | | | suis |
| | | Turicibacterales | Turicibacteraceae | Turicibacter | |
| | Clostridia | Clostridiales | Catabacteriaceae | | |
| | | | Clostridiaceae | Clostridium | hiranonis |
| | | | | | perfringens |
| | | | | Sarcina | |
| | | | Clostridiales | Eubacterium | |
| | | | FamilyXIII, | | |
| | | | IncertaeSedis | | |
| | | | Lachnospiraceae | Blautia | |
| | | | | Coprococcus | |
| | | | | Roseburia | |
| | | | | Ruminococcus | gnavus |
| Firmicutes | Clostridia | Clostridiales | Lachnospiraceae | Ruminococcus | torgues |
| Firmicutes | Clostridia | Clostridiales | Ruminococcaceae | Clostridium | orbiscindens |
| | | | | Faecalibacterium | prausnitzii |
| | | | | Oscillospira | |
| | | | Veillonellaceae | Acidaminococcus | fermentans |
| | | | | Megamonas | |
| | | | | Megasphaera | elsdenii |
| Fusobacteria | Fusobacteria | Fusobacteriales | Fusobacteriaceae | J2_29 | |
| Proteobacteria | Betaproteobacteria | Burkholderiales | Alcaligenaceae | Sutterella | |
| | Epsilonproteobacteria | Campylobacterales | Campylobacteraceae | Campylobacter | upsaliensis |
| | | | Helicobacteraceae | Flexispira | cinaedi |
| | | | | Helicobacter | muridarum |
| | Gammaproteobacteria | Aeromonadales | Succinivibrionaceae | Anaerobiospirillum | succiniciproducens |
| | | | | Succinivibrio | |
| | | Enterobacteriales | Enterobacteriaceae | Enterobacter | hormaechei |
| | | | | Escherichia | |
| | | | | Raoultella | |
| Tenericutes | Erysipelotrichi | Erysipelotrichales | Erysipelotrichaceae | Eubacterium | dolichum |
| | | | | Allobaculum | |
| | | | | Bulleidia | p_1630_c5 |
| | | | | Catenibacterium | |
| | | | | Clostridium | spiroforme |

In another aspect, the invention provides methods for treating diarrhea in an animal comprising administering to the animal a composition suitable for reducing or eliminating one or more microorganisms associated with the incidence of diarrhea in some embodiments the microorganisms associated with the incidence of diarrhea are selected from Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri, Parabacteroides distasonis, Ruminococcus gnavus, Ruminococcus torques, Streptococcus minor and Streptococcus suis.

Any composition suitable for reducing or eliminating one or more microorganisms associated with the incidence of diarrhea may be used in the present invention. In one, embodiment, the composition suitable for reducing or eliminating one or more microorganisms associated with the incidence of diarrhea comprises from about, 1 to about 20% carbohydrate; from about 3 to about 10% total dietary fiber, wherein the total dietary fiber contains from about 10 to about 40% soluble fiber and from about 90 to about 60% insoluble fiber; and from about 0.1 to about 10% omega-3 fatty acids; wherein the composition has a digestibility coefficient of at least 80. Compositions useful in the present invention are described in US Patent Publication No. 20110034411, herein incorporated by reference.

In another aspect, the invention provides methods for treating diarrhea in an animal comprising administering to the animal a composition suitable for promoting the presence of one or more microorganisms associated the absence of diarrhea. In some embodiments the microorganisms associated with the incidence of diarrhea are selected from Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri.

Any composition suitable for promoting the presence of on or more microorganisms associated with the absence of diarrhea may be used in the present invention, in one embodiment, the composition suitable for promoting the presence of one or more microorganisms associated with the absence of diarrhea comprises from about 1 to about 20% carbohydrate; from about 3 to about 10% total dietary fiber, wherein the total dietary fiber contains from about 10 to about 40% soluble fiber and from about 90 to about 60% insoluble fiber; and from about 0.1 to about 10% omega-3 fatty acids; wherein the composition has a digestibility coefficient of at least 80. Compositions useful in the present invention are describes in US Patent Publication No. 20110034411.

In various embodiments, the methods of the invention further comprise administering to the animal the composition suitable for reducing or eliminating one or more microorganisms associated with the incidence of diarrhea or suitable for promoting the presence of one or more microorganisms associated with the absence of diarrhea in combination with a probiotic, prebiotic, anti-diarrhea agent, or combination thereof.

Probiotics useful ill the present invention include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus sp., Lactobacillus paracasei, Lactobacillus rhamnosus, Lactobacillus fermentum, and Bifidobacterium sp., Enterococcus faecium and Enterococcus sp. In some embodiments, the probiotic strain is selected the group consisting of Lactobacillus reuteri (NCC2581; CNCM I-2448), Lactobacillus reuteri (NCC2592; CNCM I-2450), Lactobacillus rhamnosus (NCC 2583, CNCM I-2449); Lactobacillus reuteri (NCC2603; CNCM I-2451), Lactobacillus reuteri (NCC2613; CNCM 1-2452), Lactobacillus acidophilus (NCC2628; CNCM I-2453), Bifidobacterium adolescentis (e.g. NCC2627); Bifidobacterium sp, NCC2657 or Enterococcus faecium SF68 (NCIMB 10415). The methods of the present invention comprise administering probiotics in amounts sufficient to supply from about ¹0⁴ to about ¹⁰¹² cfu/animal/day, preferably from ¹0⁵ to about ¹⁰¹¹ cuf/animal/day, most prefrably from ¹0⁷ to ¹⁰¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390. EPI482811B1, EN1296565B1, and U.S. Pat. No. 6,929,793. In a preferred embodiment, the probiotic is Enterococcus faecium SF68 (NCIMB 10415). In one embodiment, the probiotics are encapsulated in a carrier using methods and materials known to skilled artisans.

As stated, the methods may comprise administering one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, isomaltulose, and aleurone. In one embodiment, the prebiotic is chicory root, chicory root extract, inulin, or combinations thereof. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, preferably from about 0.3 to about 7%, most preferably from about 0.5 to 5%, on a dry matter basis. The prebiotics can be integrated into the compositions using methods known to skilled artisans, e.g., U.S. Pat. No. 5,952,033.

Any anti-diarrhea agent useful for treating diarrhea can be used in the methods of the present invention. Anti-diarrhea agents useful in the present invention include, but are not limited to, ciprofloxacin, norfloxacin, bactrim, septra, trimethoprim, azithromycin, metronidazole, tormentil root extract, hyoscyamine, metoclopramide, kaolin-pectin loperamide, diphenoxylate, pancreatic lipase, and tincture of opium. Holistic anti-diarrhea drugs and compositions are also included in the invention, e.g., peppermint and ginger. The anti-diarrhea agents are administered to the animal using any method appropriate for the anti-diarrhea agent and in amounts known to skilled artisans to be sufficient to prevent or treat diarrhea.

The methods of the invention are useful for a variety of human and non-human animals including avian, bovine, canine, equine, feline, hicrine, murine, ovine, and porcine animals. In some embodiments, the animal is a companion animal such as canine or feline, preferably a dog or a cat, most preferably a cat.

All percentages expressed herein are by weight of the composition on “dry matter basis” unless specifically stated otherwise. The term “dry matter basis” means that an ingredient's percentage in a composition is measured after the moisture in the composition has been removed.

The invention is not limited to the particular methodology, protocols, and reagents described herein because they may vary. Further the terminology used herein is for the purpose of describing particular embodiments only and is not intended to limit the scope of the present invention.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a composition” or “a method” includes a plurality of such “composition” or “method.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

Unless defined otherwise, all technical and scientific terms and any acronyms used herein have the same meanings as commonly understood by one of ordinary skill in the an in the field of the invention. Although any compositions, methods, articles of manufacture, or other means or materials similar or equivalent to those described herein can be used in the practice of the present invention, the preferred, compositions, methods, articles of manufacture, or other means or materials are described herein.

All patents, patent applications, publications, and other references cited or referred to herein are incorporated herein by reference to the extent allowed by law. The discussion of those references is intended merely to summarize the assertions made therein. No admission is made that any such patents, patent applications, publications or references, or any portion thereof, are relevant prior art for the present invention and the right to challenge the accuracy and pertinence of such patents, patent applications, publications, and other references is specifically reserved.

### EXAMPLES

The invention can be further illustrated by the following examples, although it will be understood that these examples are included merely for purposes of illustration and are not intended to limit the scope of the invention unless otherwise specifically indicated.

The following food compositions were used in the Examples;

TABLE 2
Nutritional Analysis of Food Compositions Used in Study

| | Baseline | | | | | |
|---|---|---|---|---|---|---|
| | Composition | | FCA | | FCB | |
| Nutrient | % dm | g/100K | % dm | g/100K | % dm | g/100K |
| Protein | 58.90 | 12.79 | 38.72 | 8.46 | 49.15 | 10.79 |
| Fat | 29.91 | 6.50 | 26.16 | 5.71 | 28.14 | 6.18 |
| Total n3 | 3.44 | 0.75 | 0.37 | 0.08 | 1.01 | 0.22 |
| Total n6 | 4.28 | 0.93 | 6.75 | 1.47 | 4.87 | 1.07 |
| n6/n3 | 1.24 | | 18.24 | | 4.82 | |
| Carbohydrate (NFE) | 0.00 | 0.00 | 28.59 | 6.24 | 12.65 | 2.78 |
| Crude Fiber | 1.31 | 0.28 | 1.25 | 0.27 | 2.22 | 0.49 |
| TDF | 4.47 | 0.97 | 12.67 | 2.77 | 6.95 | 1.53 |
| Sol | 1.28 | 0.28 | 2.34 | 0.51 | 1.27 | 0.28 |
| Insol | 3.20 | 0.70 | 10.50 | 2.29 | 5.68 | 1.25 |

NFE = Nitrogen-free extract, determined as 100% - (% water + % protein + % fat + % ash + % crude fiber)
TDF = Total dietary fiber

The following method was used to evaluate stool quality. Stool quality was determined using a fecal scoring system with scores between 1 and 7. Normal stools are scored as a 2 or 3. A description of each score is:

Score 1—very hard and dry, requires much effort to expel from body;

Score 2—firm, but not hard; should be pliable; segmented appearance; little or no residues left on ground when picked up;

Score 3—Loglike, little or no segmentation visible; moist surface, leaves residues, but hold form when picked up;

Score 4—very moist (soggy), distinct log shape visible; leaves residues and loses form when picked up;

Score 5—very moist but has distinct shape; present in piles rather than as distinct logs; leaves residue and loses form when picked up;

Score 6—Has texture, but no defined shape; occurs as a pile or as spots; leaves residue when picked up; and

Score 7—Watery, no texture, flat; occurs as puddles.

Diarrhea is generally defined as either excessively watery feces, increased stool volume, or both. However, the threshold is not always well defined. As a general rule, a fecal score of 5 or greater on the 7 point scale is suggestive of diarrhea. Poor stool quality, which may be associated with diarrhea, is indicated by a score of 4 or greater.

### Example 1

Sixteen (16) cats with naturally occurring diarrhea were fed a commercially available canned food composition to standardize the cat's diet (“Baseline”). Then, the cats were divided into two (2) groups and fed either Food Composition A (FCA) or Food Composition B (FCB) for 4 weeks (Phase 1), During the last week of the Phase 1, diarrhea was assessed via fecal scoring, where 7=very watery; 2 to 3 is optimum; 1 is very hard and dry. Then, cats that had been fed FCA were then switched to FCB and cats that had been fed FCB were switched to FCA for another 4 weeks (Phase 2), during the last week of the Phase 2, fecal scoring was repeated. The results are shown in Table 1.

Referring to Table 1, cats FCB had significantly greater improvement in fecal score, compared to those FCA. Other data show that for eats fed FCA, only 12.5% of cats developed normal stools. In contrast, 43.8% of cats fed FCB developed normal stools (defined as a fecal score of 2 or 3).

TABLE 3
Fecal Scores

| Time | Group | Product | N | Mean | Std. Error |
|---|---|---|---|---|---|
| Baseline | 1 | Control | 9 | 5.7972 | 0.4771 |
| Phase 1 | 1 | Diet A | 9 | 4.8656 | 0.4771 |
| Phase 2 | 1 | Diet B | 9 | 3.8806 | 0.4771 |
| Baseline | 2 | Control | 7 | 5.0873 | 0.541 |
| Phase 1 | 2 | Diet B | 7 | 3.6489 | 0.541 |
| Phase 2 | 2 | Diet A | 6 | 4.199 | 0.5576 |

Extraction of DNA from Feline fecal Samples: Fecal material ranging in weight from 0.03 to 0.2 grams were placed into 15 ml conical tubes containing 1.5 ml PBS (0.85% NaCl, 120 mM NaH2PO4, pH=8.0). The samples were vortexed to uniformly suspend the fecal material. The slurry was pelleted by centrifugation at 5525×g (5100 rpm) for 10 minutes, Supernatants were poured off to leave as little liquid as possible. The pellets were resuspended in 0.5 ml Lysis solution (0.15M NaCl. 0.1 M EDTA, pH=8.0) containing 15 mg/ml of Lysozyme. The samples were then incubated in a 37° C. shaking water bath for 30 minutes with constant agitation. 0.5 mls of STS Solution (0.1 M NaCl, 0.48M Tris HCl [pH=8.0], 10% SDS, was added to each sample and they again were incubated in a shaking 37° C. water bath for 30 minutes with constant agitation. Three cycles of freezing in a −80° C. freezer for 10 minutes, and thawing in warm water, were performed to break open the bacterial cell walls. After the third thaw, 1.5 μl of Proteinase K (Fisher Scientific, Pittsburgh, Pa.) was added to each sample to a final concentration of 50 μg/ml and incubated in a 37° C. shaking water bath for 30 minutes with constant agitation. Samples were transferred to 2.0 ml micro centrifuge tubes and centrifuged at 17,500×g (14,000 rpm) for 20 minutes in refrigerated micro centrifuge. 850 μl of the Supernatant was removed, without disturbing the pellet, and placed into a clean tube on ice. Samples stay on ice for remainder of the processing. An equal volume of Phenol pH=7.9 (Fisher Scientific, Pittsburgh, Pa.) was added and the samples which were then mixed by repeatedly inverting the tubes for 30 seconds. The mixture was centrifuged at 16,500×g (12,500 rpm) for 6 minutes, 700 μl of the Supernatant was removed and placed in a clean micro centrifuge tube. An equal volume of Phenol/Chloroform/Isoamyl alcohol (25:24:1) was added and the samples, which were then mixed by repeatedly inverting the tubes for 30 seconds. The mixture was centrifuged at 16,500×g for 6 min. 550 μl of the Supernatant was removed and placed in a clean micro centrifuge tube. An equal volume of Chloroform/Isoamyl alcohol (24:1) was added and the samples, which were then mixed by repeatedly inverting the tubes for 30 seconds. The mixture was centrifuged at 16,500×g for 6 min, 400 μl of the Supernatant was removed and placed in a clean micro centrifuge tube, 400 μl Ice Cold isopropanol and 960 μl 10.5M Ammonium Acetate (1.125M final concentration) were added and the samples were mixed by repeatedly inverting the tubes for 30 seconds, then placed in a −80° C. freezer Overnight or until processing (up to 1-2 weeks). The DNA pellets were obtained by centrifuging at 17,500×g (14,000 rpm) for 20 min in refrigerated micro centrifuge. The supernatants were carefully poured off and the pellets were washed with 500 μl 70% Ethanol. The Ethanol was gently poured off, and the pellets allowed to air dry until all samples were completed. Pellets were then dried under vacuum for 5 minutes at Room Temperature. The pellets, ranging from small and clear to large and brownish in color, were re-suspended in 200 μl TE Buffer (10 mM Tris HCl [pH=8.8]. 1 mM EDTA, pH=8.0). All samples were then stored at −80° C. until further analysis, Samples were quantified by Qua/It™ (Invitrogen, Carlsbad, Calif.).

16S rDNA pyrosequencing: PCR products were sent to Core for Applied Genomics and Ecology (University of Nebraska-Lincoln, USA) where equal amount of each were pooled and sequenced by the 454 GS-FLX Pyrosequencer. (Roche, Branford, Conn.). The V1-V2 region of the 16S rRNA gene was amplified using bar-coded fusion primers with the 454 A or B titanium sequencing adapters followed by a unique 8-base barcode sequence (B) and finally the 5′ ends of primer A-8FM CCATCTCATCCCTGCGTGTCTCCGACTCAGBBBBBBBBAGAGTTTGATCMTGGCTCA G) and of primer B-357R (5′-CCTATCCCCTGTGTGCCTT-GGCAGTCTCAGBBBBBBBBBCTGCTGCCTYCCGTA.

3′). All PCR reactions were quality controlled for amplicon saturation by gel electrophoresis; band intensity was quantified against standards using GeneTools software (Syngene, Frederick, Md.). For each region of a two-region picotiter plate, amplicons from 48 reactions were pooled in equal amounts and gel purified. The resulting products were quantified using PicoGreen (Invitrogen, Carlsbad, Calif.) and a Qubit fluorometer (invitrogen, Carlsbad, Calif.) before sequencing using 454 GS FLX titanium chemistry.

Data Processing Pipeline: The raw sequencing data from 454 pyrosequencing machine were processed using the Quantitative Insights Into Microbial Ecology (QIIME) workflow. Specifically, the data were first subject to the quality filters to remove low quality reads. These quality criteria include: 1). A complete barcode sequence immediately followed by a forward primer sequence, with no mismatches allowed. 2) Read lengths are between 200 and 1000 bases. 3) Average quality score for each read is 25 or higher using the QUAL file. 4) Maximum length of homopolyer run is 6. The processed reads were then de-multiplexed into barcode-indexed sample categories. The barcode, forward primer and reverse primer were subsequently trimmed from each read. We have obtained a total of 555,366 reads for 48 samples with an average of 11,591 reads per sample. The average length for the reads was 358 bases. Reads were clustered into Operational Taxonomic Units (OTU's) using reference based uclust algorithm where similarity threshold was set at 97%. The reference data the contained the most recent greengenes OTUs that were derived from the Greengenes databases (http://greengenes.lbl.gov). A consensus taxonomic lineage was assigned to each OTU using the new greengenes taxonomy (http://greengenes.lbl.gov). Finally, an OTU table which is a matrix of OTU by sample with proper taxonomic identifications for each OTU was constructed.

Microbiome analysis of fecal samples detected 146 species, 96 genera, 47 families, 25 orders, 14 classes, and 8 phyla in the 48 samples sequenced. Analysis of the data was conducted at the species level. Orthogonal Partial least square method was applied using the software package SIMCA-P+ (version 12.0.1.0, Umetrics AB, Umeá, Sweden) and MATLAB (The Math Works Inc., Natick, Mass., USA) routines, Significant correlations between the microbiome and fecal scores were found for FCB at the species level as well as for FAC. List of significant bacteria with R>±0.2 (P=0.05) are shown in Table 4.

TABLE 4
Significant Bacteria Identified

| Order | Family | Genus | Species | FCA | FCB | Combined |
|---|---|---|---|---|---|---|
| Coriobacteriales | Coriobacteriaceae | Collinsella | | −0.508 | −0.59668 | −0.59668 |
| Coriobacteriales | Coriobacteriaceae | Collinsella | aerofaciens | −0.36093 | | −0.360928 |
| Coriobacteriales | Coriobacteriaceae | Stackia | | −0.4137 | −0.82564 | −0.82564 |
| Bacteroidales | Bacteroidaceae | Bacteroides | | | −0.46126 | −0.46126 |
| Bacteroidales | Bacteroidaceae | Bacteroides | coprocola | | 0.49824 | 0.49824 |
| Bacteroidales | Bacteroidaceae | Bacteroides | coprophilus | | −0.48589 | −0.48589 |
| Bacteroidales | Bacteroidaceae | Bacteroides | plebeius | 0.382086 | −0.46366 | |
| Bacteroidales | Porphyromonadaccae | Parabacteroides | | 0.297934 | 0.21864 | 0.297934 |
| Bacteroidales | Porphyromonadaccae | Parabacteroides | distasonis | | 0.25860 | 0.25860 |
| Bacteroidales | Prevotellaceae | Prevotella | | −0.45716 | 0.47125 | |
| Bacteroidales | Prevotellaceae | Prevotella | copri | −0.50019 | | −0.500186 |
| Bacteroidales | Rikenellaceae | Alistipes | | −0.27001 | | −0.270008 |
| Bacteroidales | Rikenellaceae | Alistipes | putredinis | 0.290766 | | 0.290766 |
| Lactobacillales | Lactobacillaceae | Lactobacillus | | 0.24183 | 0.23151 | 0.23151 |
| Lactobacillales | Lactobacillaceae | Lactobacillus | helveticus | 0.344703 | | 0.344703 |
| Lactobacillales | Lactobacillaceae | Lactobacillus | saerimneri | 0.252733 | | 0.252733 |
| Lactobacillales | Streptococcaceae | Streptococcus | | 0.21118 | 0.29202 | 0.29202 |
| Lactobacillales | Streptococcaceae | Streptococcus | minor | | 0.32565 | 0.32565 |
| Lactobacillales | Streptococcaceae | Streptococcus | suis | 0.544654 | | 0.544654 |
| Turicibacterales | Turicibacteraceae | | | 0.282503 | | 0.282503 |
| Turicibacterales | Turicibacteraceae | Turicibacter | | −0.34312 | | −0.34312 |
| Clostridiales | Calabacteriaceae | | | | −0.53453 | −0.53453 |
| Clostridiales | Clostridiaceae | | | 0.471341 | | 0.471341 |
| Clostridiales | Clostridiaceae | Clostridium | | 0.359129 | 0.20388 | 0.359129 |
| Clostridiales | Clostridiaceae | Clostridium | hiranonis | −0.29325 | −0.20820 | −0.293245 |
| Clostridiales | Clostridiaceae | Clostridium | perfringens | 0.438655 | −0.29590 | 0.438655 |
| Clostridiales | Clostridiaceae | Sarcina | | 0.4528 | 0.45716 | 0.45716 |
| Clostridiales | Clostridiales Family | | | 0.313387 | | 0.313387 |
| | XIII IncertacSedis | | | | | |
| Clostridiales | Clostridiales Family | Eubacterium | | −0.2683 | −0.66392 | −0.66392 |
| | XIII IncertacSedis | | | | | |
| Clostridiales | Lachnospiraceae | | | | −0.76376 | −0.76376 |
| Clostridiales | Lachnospiraceae | Blautia | | −0.4164 | −0.62680 | −0.62680 |
| Clostridiales | Lachnospiraceae | Coprococcus | | −0.4698 | −0.54182 | −0.54182 |
| Clostridiales | Lachnospiraceae | Roseburia | | | −0.62354 | −0.62354 |
| Clostridiales | Lachnospiraceae | Ruminococcus | | −0.3189 | −0.37107 | −0.37107 |
| Clostridiales | Lachnospiraceae | Ruminococcus | gnavus | 0.388937 | | 0.388937 |
| Clostridiales | Lachnospiraceae | Ruminococcus | torques | | 0.45208 | 0.45208 |
| Clostridiales | Ruminococcaceae | | | −0.432 | −0.21820 | −0.432 |
| Clostridiales | Ruminococcaceae | Clostridium | orbiscindens | | −0.59092 | −0.59092 |
| Clostridiales | Ruminococcaceae | Faecalibacterium | prausnitzil | | 0.32644 | 0.32644 |
| Clostridiales | Ruminococcaceae | Oscillospira | | | 0.38267 | 0.38267 |
| Clostridiales | Veillonellaceae | | | 0.425241 | 0.26287 | 0.425241 |
| Clostridiales | Veillonellaceae | Acidaminococcus | fermentans | | −0.66154 | −0.66154 |
| Clostridiales | Veillonellaceae | Megamonas | | 0.50935 | | 0.50935 |
| Clostridiales | Veillonellaceae | Megasphaera | elsdenii | 0.24844 | −0.54466 | −0.54466 |
| Fusobacteriales | Fusobacteriaceae | J2_29 | | −0.21923 | | −0.219228 |
| Burkholderiales | Alcaligenaceae | Sutterella | | −0.40143 | 0.29011 | |
| Campylobacterales | Campylobacteraceae | Campylobacter | upsaliensis | | −0.78506 | −0.78506 |
| Campylobacterales | Helicobacteraceae | Flexispim | cinaedi | 0.453162 | −0.32182 | |
| Campylobacterales | Helicobacteraceae | Helicobacter | | 0.311453 | | 0.311453 |
| Campylobacterales | Helicobacteraceae | Helicobacter | muridanum | 0.373168 | | 0.373168 |
| Aeromonadales | Succinivibrionaceae | | | 0.39914 | −0.70980 | |
| Aeromonadales | Succinivibrionaceae | Anacrobiospirillum | succiniciproducens | | 0.20354 | 0.20354 |
| Aeromonadales | Succinivibrionaceae | Succinivibrio | | 0.570433 | | 0.570433 |
| Enterobacteriales | Enterobacteriaceae | | | 0.299915 | | 0.299915 |
| Enterobacteriales | Enterobacteriaceae | Enterobacter | hormaechei | 0.303502 | | 0.303502 |
| Enterobacteriales | Enterobacteriaceae | Escherichia | | 0.25593 | −0.27550 | |
| Enterobacteriales | Enterobacteriaceae | Raoultella | | 0.37943 | −0.71560 | −0.71560 |
| Erysipelotrichales | Erysipelotrichaceae | | | | 0.45821 | 0.45821 |
| Erysipelotrichales | Erysipelotrichaceae | Eubacterium | dolichum | −0.331 | −0.49316 | −0.49316 |
| Erysipelotrichales | Erysipelotrichaceae | Allobaculum | | −0.25378 | | −0.25378 |
| Erysipelotrichales | Erysipelotrichaceae | Bulleidia | p_1630_c5 | | 0.35304 | 0.35304 |
| Erysipelotrichales | Erysipelotrichaceae | Catenibacterium | | 0.302167 | | 0.302167 |
| Erysipelotrichales | Erysipelotrichaceae | Clostridium | spiroforme | −0.3946 | 0.51251 | 0.51251 |

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention mar be practiced otherwise than as specifically described.

What is claimed is:

1. A method for diagnosing impending diarrhea in an asymptomatic animal comprising:
obtaining a feral sample from the animal;
identifying one or more microorganisms in the fecal sample, and
diagnosing impending diarrhea in the animal based on (1) the presence of one or microorganisms selected from a first group consisting of Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri. Bulleidia p_{—}1630_c5, Parabacterides distasonis, Ruminococcus gnavus, Ruminococcus torques, Streptococcus minor, and Streptococcus suis, (2) the absence of one or more of the microorganisms selected from a second group consisting of Acidaminococcus fermentans, Bacteroides coprophilus, Campylobacter upsaliensis, Clostridium hiranonis, Clostridium orbiseindens, Collinsella aerofaciens, Eubacterium dolichum, Megasphaera elsdenii, and Prevotella copri, or a combination thereof, or (3) a combination thereof.

2. The method of claim 1 wherein the animal is diagnosed to have impending diarrhea if (1) two, three, four, five or more microorganisms selected from a first group consisting of Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri, Bulleidia p_{—}1630_c5, Parabacterides distasonis, Ruminococcus gnavus, Ruminococcus torques, Streptococcus minor, and Streptococcus suis are present in the fecal sample, (2) two, three, four, five or more microorganisms selected from a second group consisting of Acidaminococcus fermentans, Bacteroides coprophilus, Campylobacter upsaliensis, Clostridium hiranonis, Clostridium orbiscindens, aerofaciens, Eubacterium dolichum, Megasphaera elsdenii, and Prevotella copri are absent from the fecal sample, or 3 combinations thereof.

3. The method of claim 1 wherein the animal is diagnosed to have impending diarrhea if (1) at least three microorganisms selected from a first group consisting of Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri. Bulleidia p_{—}1630_c5, Parabacterides distasonis, Ruminococcus gnavus, Ruminococcus torques, Streptococcus minor, and Streptococcus suis are present in the fecal sample, (2) at least three microorganisms selected from a second group consisting of Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri are absent from the fecal sample, or (3) combinations thereof.

4. The method of claim 1 wherein the microorganisms are selected from the first group consisting of Alistipes putredinis, Anaerobiospirillum succiniciproducens, Bacteroides coprocola, Clostridium perfringens, Clostridium spiroforme, Enterobacter hormaechei, Faecalibacterium prausnitzii, Helicobacter muridarum, Lactobacillus helveticus, Lactobacillus saerimneri. Bulleidia p_{—}1630_c5, Parabacterides distasonis, Ruminococcus gnavus, Ruminococcus torques, Streptococcus minor, and Streptococcus suis.

5. The method of claim 1 wherein the microorganisms are selected from the first group consisting of Bacteroides coprocola, Clostridium perfringens, Clostridium spiroforme, Enterobacter hormaechei, Faecalibacterium prausnitzii. Helicobacter muridarum, Lactobacillus helveticus, Bulleidia p_{—}1630_c5, Ruminococcus gnavus, Ruminococcus torques, Streptococcus minor, and Streptococcus suis.

6. The method of claim 1 wherein the microorganisms are selected from the first group consisting of Clostridium spiroforme and Streptococcus suis.

7. The method of claim 1 wherein the microorganisms are selected from the second group consisting of Acidaminococcus fermentans, Bacteroides coprophilus, Campylobacter upsaliensis, Clostridium hiranonis, Clostridium orbiscindens, Collinsella aerofaciens, Eubacterium dolichum, Megasphaera elsdenii, and Prevotella copri.

8. The method of claim 1 wherein the microorganisms are selected from the second group consisting of Acidaminococcus fermentans, Campylobacter upsaliensis, Clostridium orbiscindens, Megasphaera elsdenii, and Prevotella copri.

9. The method of claim 1 wherein the microorganisms are selected from the second group consisting of Acidaminococcus fermentans and Campylobacter upsaliensis.

10. The method of claim 1 wherein the animal is a feline.


SEQUENCE LISTING (2 sequences)

SEQ ID NO: 1 - DNA, 58 bp, Artificial Sequence
  Primer for amplification of the V1-V2 region of the 16S rRNA gene containing 454 A or B titanium sequencing adapters followed by a unique 8-base barcode sequence and the 5′ ends of primer A-8FM
ccatctcatccctgcgtgtctccgactcagnnnnnnnnagagtttgatcmtggctcag

SEQ ID NO: 2 - DNA, 53 bp, Artificial Sequence
  Primer for amplification of the V1-V2 region of the 16S rRNA gene containing the 454 A or B titanium sequencing adapters followed by a unique 8-base barcode sequence and the 5′ ends of primer B-357R
cctatcccctgtgtgccttggcagtctcagnnnnnnnnctgctgcctyccgta